(19) United States
(12) Patent Application Publication
(10) Pub. No.: US 2005/0176059 A1
(43) Pub. Date: Aug. 11, 2005

(54) Bio-safe dispenser and optical analysis disc assembly

(76) Inventors: Andrew Attila Pal, Rancho Santa Margarita, CA (US); Michael Craig Browne, Lake Forest, CA (US); Horacio Kido, Niland, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/347,119

(22) Filed: Jan. 17, 2003

Related U.S. Application Data
(60) Provisional application No. 60/354,012, filed on Jan. 31, 2002.

(51) Int. Cl. C12M 1/34; G01N 33/53

(52) U.S. Cl. 435/7.1; 435/287.2

(57) ABSTRACT

A liquid sample dispenser for use with an optical analysis disc. The dispenser includes a body portion, a tip portion connected to the body portion, first and second retainer members moveably supported within the tip portion, and a sealant contained between the first and second retainer members. One of the retainer members also functions as an inlet stopper. The associated optical analysis disc includes a substrate and a channel layer associated with the substrate. The disc further includes a cover portion located so that the channel layer is positioned between the substrate and the cover portion. An inlet port formed in the cover portion is adapted to receive a test sample from the tip portion of the dispenser. The sample is loaded form the dispenser into the disc followed by the inlet stopper and sealant to seal the sample in the disc. Related methods for loading a biological sample into the analysis disc through the dispenser, and sealing the disc inlet are also provided. Kits including the disc and dispenser or the disc and one-use dispenser tips are also provided.

### CROSS-REFERENCE TO RELATED APPLICATIONS

This application claims the benefit of priority from U.S. Provisional Application Ser. No. 60/354,012 filed Jan. 31, 2002 which is herein incorporated by reference in its entirety.

### BACKGROUND OF THE INVENTION

1. Field of the Invention

This invention relates in general to the loading of a biological sample to be analyzed on the correct locations of a disc. More specifically, but without restriction to the particular embodiments hereinafter described in accordance with the best mode of practice, this invention relates to a bio-safe dispenser and a related optical analysis disc assembly.

2. Discussion of the Background Art

An optical bio-disc is commonly used to conduct analysis on biological samples. The optical bio-disc operates in conjunction with a computer system provided with an optical disc drive and a display monitor.

The principal structural elements of an optical bio-disc include a cap portion, an adhesive member or channel layer, and a substrate. The cap portion includes one or more inlet ports for the loading of samples on the disc in order to be analyzed. The main biological hazard in loading samples in liquid form onto or into a disc, comes from the potentially infectious residues that may be spilled onto the exterior of the disc from the area adjacent the inlet port.

Furthermore, another risk is the possible cross contamination of different samples and/or loading a particular sample into a wrong analysis chamber. The present invention provides methods and apparatus including a dispenser and optical analysis disc that effectively address these concerns to thereby provide a useful and safe sample dispenser and a related optical analysis disc assembly.

### SUMMARY OF THE INVENTION

The present invention is directed to a bio-safe dispenser and optical analysis disc assembly. The present dispenser may be implemented in the tip of a pipette.

More particularly, the present invention is directed to a liquid sample dispenser having a body portion, a tip portion connected to the body portion, first and second retainer members moveably supported within the tip portion, and a sealant contained between the first and second retainer means.

According to one aspect of this invention, there is provided an optical analysis disc for use with the dispenser. In one embodiment of the dispenser, the tip portion includes an operational end adapted to interact with the optical analysis disc. In a more specific embodiment thereof, the operational end is open.

In accordance with one preferred implementation of this invention, the first retainer member is positioned proximal to the operational end of the tip portion and includes a sealing plug adapted to seat in an inlet port the optical analysis disc.

As provided by yet another aspect of this invention, the operational end may be advantageously provided with coded indicia which correspond to counterpart indicia associated with a respective inlet port in the optical analysis disc so that a sample in the tip portion may be loaded into the channel layer only through the respective inlet port having the counterpart indicia.

In other preferred embodiments of this invention, the inlet port is in fluid communication with the channel layer. In alternate embodiments, the inlet port in the disc may include an initial break-away seal to protect any pre-loaded chemistries provided in the channel layer during delivery and storage of the disc.

According to still yet another aspect of this invention, there is provided an optical analysis disc including a substrate, a channel layer associated with the substrate, a cover portion, and an inlet port formed in the cover portion adapted to receive a test sample and a sealing plug. The channel layer is preferably positioned between the substrate and the cover portion.

In accordance with still further aspects of the present invention, there is provided an analysis kit. This kit is advantageously provided with a dispenser having a body portion, a tip portion connected to the body portion, first and second retainer members moveably supported within the tip portion, and a sealant contained between the first and second retainer means. The kit further includes an optical analysis disc including a substrate, a channel layer associated with the substrate, a cover portion located so that the channel layer is positioned between the substrate and the cover portion, and an inlet port formed in the cover portion adapted to receive a test sample and one of the retainer members.

In one preferred embodiment of the analysis kit, the inlet port is in fluid communication with the channel layer. In alternate embodiments, the inlet port in the disc may include an initial break-away seal to protect any pre-loaded chemistries provided in the channel layer during delivery and storage of the analysis kit. The first retainer member may be located proximal to an open end of the tip portion and be implemented as a sealing plug adapted to seat in the inlet port.

The tip portion of the dispenser provided in the present analysis kit preferably includes an operational end adapted to interact with the optical analysis disc. In a more specific embodiment, the operational end is open.

In an alternate preferred embodiment of the present invention, the first retainer member of the dispenser provided in the analysis kit is positioned proximal to the operational end of the tip portion and implemented as a sealing plug adapted to seat in the inlet port of the optical analysis disc.

And according to still yet an additional aspect of the analysis kit of this invention, the operational end of the dispenser may include coded indicia which correspond to counterpart indicia associated with a respective inlet port in the optical analysis disc so that a sample in the tip portion may be loaded into the channel layer only through the respective inlet port having the counterpart indicia.

In any of the above discussed embodiments of the dispenser according to this invention, the tip portion thereof may advantageously include a disinfectant and/or include pre-loaded antigens, agents, reagents, or other assay solutions. These pre-loaded assay solutions are preferably provided in a separate tip unit with the pre-loaded sealant. The separate tip unit is then attached to the pipette or dispenser by a threaded connection or other suitable quick release or adapter mechanisms.

The present invention is also directed to a method for loading a biological sample on a analysis disc through a dispenser having a tip portion including retainer means and a sealant. This method includes the steps of activating the dispenser for retrieving an pre-measured amount of the sample in the tip portion; directing the tip portion to a disc having an inlet port in communication with a channel layer; placing the tip portion within the inlet port; activating the dispenser for moving the sample into the channel layer; and sealing the inlet port.

This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 20, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 17, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6, 2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; and U.S. patent application Ser. No. 10/307,263 entitled “Magneto-Optical Bio-Discs and Systems Including Related Methods” filed Nov. 27, 2002. All of these applications are herein incorporated by reference in their entireties. They thus provide background and related disclosure as support hereof as if fully repeated herein.

The above described methods and apparatus according to the present invention as disclosed herein can have one or more advantages which include, but are not limited to, simple and quick on-disc processing without the necessity of an experienced technician to run the test, small sample volumes, use of inexpensive materials, and use of known optical disc formats and drive manufacturing. These and other features and advantages will be better understood by reference to the following detailed description when taken in conjunction with the accompanying drawing figures.

### BRIEF DESCRIPTION OF THE DRAWINGS

FIG. 1 is a pictorial elevation view of a dispenser and an optical analysis disc according to the present invention;

FIGS. 2A and 2B are views depicting operation of the present dispenser retrieving a liquid test sample;

FIG. 3 is a pictorial elevation view of the dispenser loading the liquid sample into the optical analysis disc;

FIG. 4 is a view illustrating sealing of the inlet port of the optical analysis disc;

FIG. 5 is a pictorial elevation view of a different embodiment of a dispenser according to the present invention;

FIGS. 6A and 6B are perspective views of two different embodiments of the tip portion of the present invention each including an alternate raised physical coding key according to certain aspects hereof;

FIG. 7 is a longitudinal cross sectional view of the tip portion shown in FIG. 6B;

FIG. 8 is a collection of various embodiments of the raised physical coding key according to the present invention;

FIG. 9A is a perspective view of a dispenser and disc according to the present invention wherein the dispenser tip is provided with a physical coding key and the disc is provided with corresponding counterpart indicia;

FIG. 9B is a perspective view of a consumer level product implementation of a kit according to the present invention that includes a pre-loaded disc and a set of related dispenser tips pre-loaded with different agents, reagents, or other chemistries for conducting several different assays on the single disc as illustrated in a bio-safe jewel case;

FIG. 10A is a across sectional detail elevation view of the dispenser and optical analysis disc of FIG. 9 illustrating association of the dispenser tip with the disc just before loading of the liquid sample, stopper plug, and sealant; and

FIG. 10B is a view similar to FIG. 10A showing the liquid sample loaded in the disc and the inlet port thereof sealed according to the present invention.

### DETAILED DESCRIPTION OF THE INVENTION

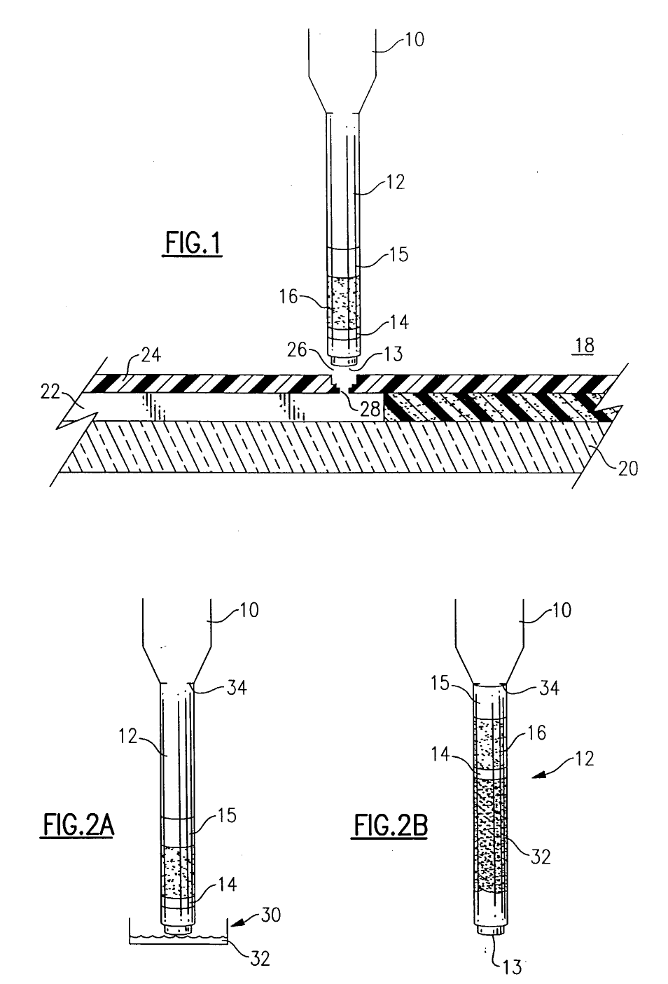

With reference now to FIG. 1, there is shown a dispenser or pipette 10 including a tip portion 12. The tip 12 is provided with an opening 13. The pipette tip 12 is pre-loaded with two rubber stoppers 14 and 15 having positioned therebetween a sealant or adhesive 16 such as “crazy glue”. The lower stopper 14 also serves as a plug as described in further detail below. The sealant 16 is preferably of the type that is liquid while unexposed to air and quickly cures when exposed to air.

Also shown in FIG. 1 is a schematic representation of an optical analysis disc such as those discussed in the above referenced commonly assigned co-pending United States Patent Applications. As principal components, this type of disc 18 includes a substrate layer 20, a channel layer 22, and a top cover segment 24. As illustrated in FIG. 1, the top cover 24 is provided with an inlet port 26 adapted to receive tip 12 of pipette 10. The inlet port 26 provides open fluid communication to the chamber layer 22, as illustrated. In one preferred embodiment, inlet port 26 includes an annular lip portion 28 adapted to snuggly received the rubber stopper or plug 14. In an alternate embodiment, the inlet port 26 may include a break-away seal to protect any pre-loaded chemistries provided in the channel layer 22 during delivery and storage of the disc.

With reference next to FIGS. 2A and 2B, a method according to the present invention will be described in further detail. As illustrated in FIG. 2A, the pipette tip 12 is directed to a container 30 including a liquid sample 32. As shown in FIG. 2B, the pipette 10 is activated to retrieve an amount of sample 32 in the pipette tip 12 as illustrated. The lower rubber stopper 14, sealant 16, and upper stopper 15 move in register upwardly in the pipette tip until stopped by a stopping ring 34. Thus as the tip 12 is loaded with liquid sample 32, movement of the upper rubber stopper 15 is eventually stopped by the stopping ring 34. In this manner, the pipette tip may be loaded with a pre-measured amount of sample. This is achieved by adjusting the location of the stopping ring 34 relative to the size of the stoppers 14 and 15 and the available open volume in the tip after the volume of the stoppers and sealant has been accounted for.

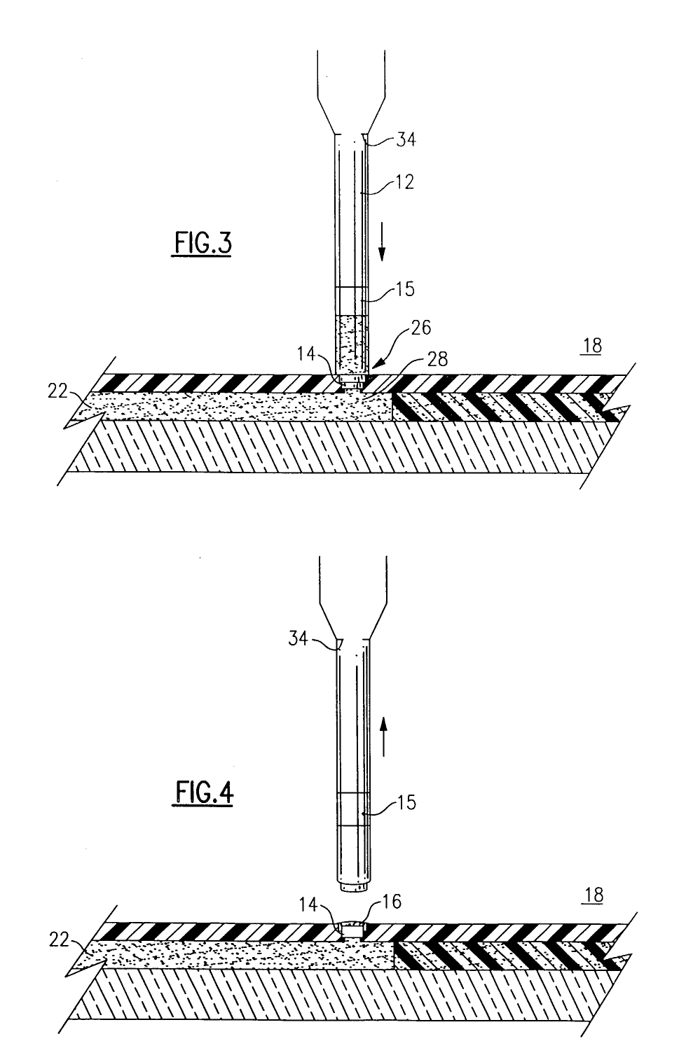

Referring now to FIG. 3, the loaded pipette tip is then directed to the disc 18. The end of the pipette tip 12 is situated within the inlet port 26. Thereafter the pipette is activated to move the sample into the channel layer 22. With further activation of the dispenser, the lower rubber stopper 14 is placed within the inlet port 26 and snugly seated on the annular lip portion 28. As illustrated in FIG. 4, with further application of the pipette, the sealant 16 is released over the seated stopper 14 to thereby provide a leak tight seal between the external environment of the disc 18 and the channel or chamber layer 22. The loaded disc with the desired sample safely sealed therein is then in condition for use in a bio-drive such as those described in the U.S. patent applications referenced above.

Disinfecting capabilities are another bio-safe aspect relating to the dispenser 10 of the present invention. The main biological hazard of transferring sample liquids 32 from the dispenser 10 to the disc 18 comes from the potentially infectious residues that may be spilled onto the exterior of the disc 18 from the area adjacent the inlet port 26. The high reactivity of cyanoacrylates is expected to have a sterilizing effect upon the liquid sample 32 that it comes in contact with. However, additives in the cyanoacrylate adhesive would enhance the adhesive's microbicidal qualities. These would have to be slightly acidic agents in order as to not interfere with the normal anionic polymerization reaction the cyanoacrylates undergo upon exposure to moisture and weak bases. Disinfectants that fall under this category may be aromatic in nature and include triclosan, phenol, and trichlorophenol. In one preferred embodiment, the bio-safe dispenser 10 is implemented in a positive displacement type pipette system. This is analogous to a system using a syringe plunger. Positive displacement systems are preferred, rather than standard negative displacement pipettes (e.g. Rainin or Eppendorf pipettes), such systems being able to generate additional vacuum to “pull in” the system of plugs 14 and 15 as well as the sealant or adhesive 16. To reduce the degree of vacuum needed to move the plugs 14 and 15, and adhesive 16 system within the tip 12, the cyanoacrylate adhesive is preferably formulated with lower boiling point solvents so that bubbles would not form when pulling on the syringe plunger.

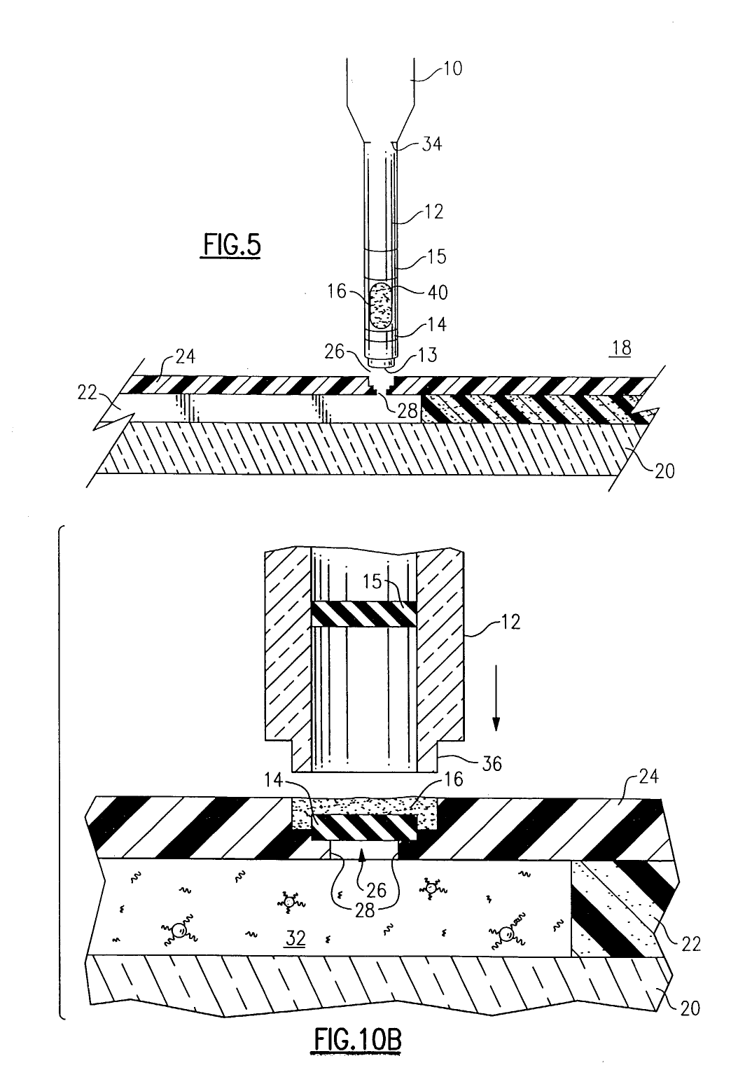

Another method of reducing the fluid drag effects resulting form zero velocity between the sealant 16 and the inner wall of the tip 12 is to pre-load the sealant or adhesive 16 in a capsule 40 as illustrated in FIG. 5, that shows a different embodiment of a dispenser according to the present invention.

According to such embodiment, the capsule may be formed of any suitable material which provides low friction and is easily dissolved or broken when released from the tip 12 during the sealing operation.

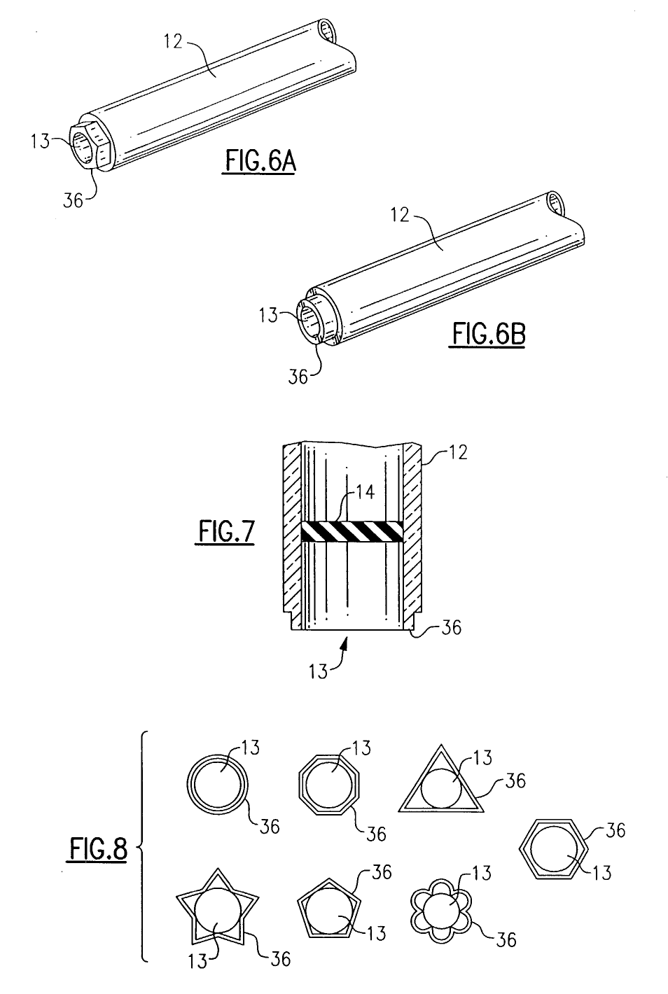

Additional aspects of the present invention will now be discussed in connection with FIGS. 6A to 10B. These aspects are directed to an additional safety feature implemented on disc and dispenser assemblies when the disc includes several analysis chambers. In such a disc, the different chambers may include different agents, reagents, or other preloaded chemistries for conducting several different assays on a single disc. In some cases, these multi-assay discs may require the use of different dispensers or pipettes, each used for a particular and separate sample. With these aspects of the present invention, cross contamination of different samples and loading a particular sample into the wrong analysis chamber is prevented.

FIGS. 6A and 6B are perspective views of two different embodiments of the tip portion 12 each including an alternate raised physical coding key 36. These coding keys may be integrally formed as a part of the tip 12 from the same material. Alternatively, the coding key 36 and tip 12 may be provided as a separate unit with pre-loaded sealant or preloaded sealant and assay solution. The separate tip unit is then attached to the pipette or dispenser 12 by a threaded connection or other suitable connection methods. FIG. 7 illustrates a longitudinal cross sectional view of the tip portion 12 shown in FIG. 6B. In one embodiment of the present invention, several different tips 12 are provided in a single kit for performing any number or related or desired assays. Each tip 12 may be pre-loaded with a disinfectant and/or include different pre-loaded antigens, agents, reagents, or other assay solutions as desired.

With reference now to FIG. 8, there is shown, by way of example and not limitation, a collection of various embodiments of the raised physical coding key 36. These shapes include for example, but are not limited to, circles, octagons, triangles, stars, pentagons, or clovers.

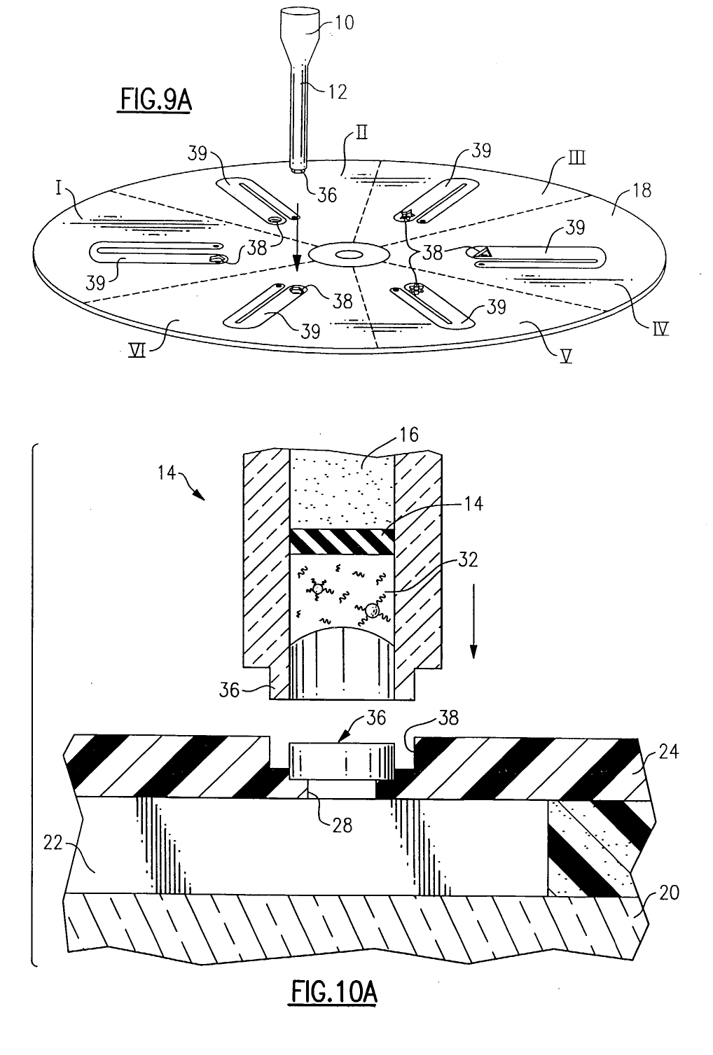

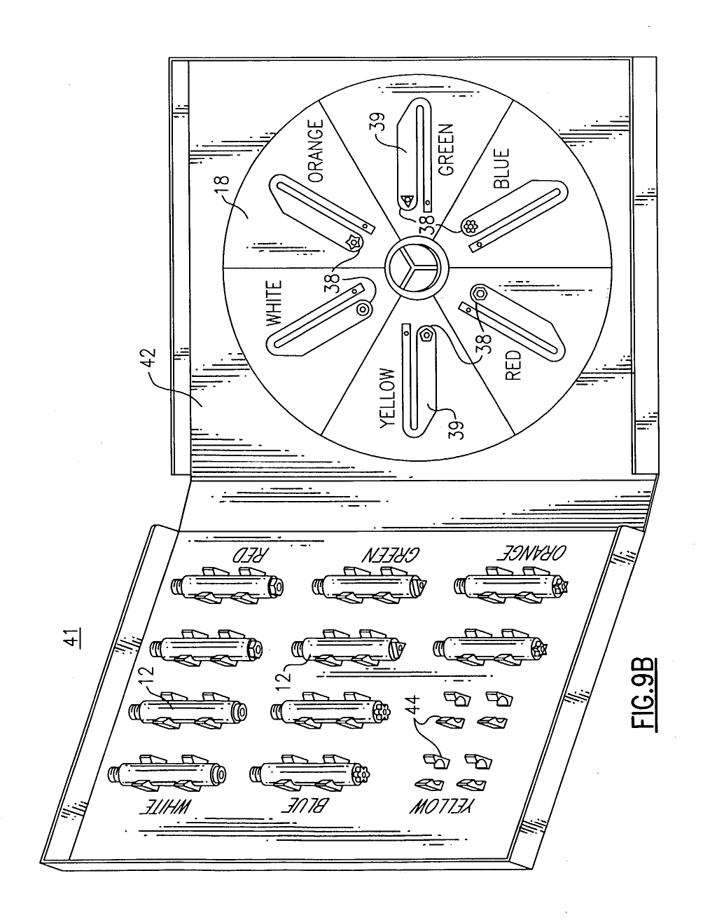

In FIG. 9A there is shown a perspective view of the dispenser 10 and disc 18. The dispenser tip 12 is provided with a physical coding key 36 and the disc is provided with corresponding counterpart indicia 38. As illustrated, the counterpart indicia 38 if formed by making a recessed shape in the top cap 24 that corresponds to the shape of the code key 36. In this manner, a lock and key mating is achieved. In addition to this lock and key mating system, the present invention may also include a color-coding safety feature. In this specific embodiment of the present invention, disc 18 including several analysis chambers or channels 39 is divided into triangular-shaped segments I, II, III, IV, V, and VI. Each of these segments I, II, III, IV, V, and VI may include unique identifying indicia such as different colors, different pictograms, different visual designs, or written language in different fonts and/or colors, for example. In this manner, the tips supplied with the disc are preferably “color-coded” or “indicia-coded” to correspond to the analysis chamber 39 containing compatible pre-loaded chemistries.

FIG. 9B is a perspective view of a consumer level product implementation of a kit 41 according to the present invention. The kit 41 includes a pre-loaded disc 18 and a set of related dispenser tips 12 pre-loaded with different agents, reagents, or other chemistries for conducting several different assays on a respective single disc 18. As illustrated, the kit components including the disc 18 and the set of dispenser tips 12 are preferably provided in a bio-safe jewel case 42. As also shown each of the tips 12 is retained in the jewel case 42 by use of detents 44. As further illustrated in FIG. 9B the disc 18 has been color-coded according to one safety aspect of this invention. Each of the different analysis chambers 39 are situated in a different color as indicated. This includes, by way of example and not limitation, the red, blue, green, orange, white, and yellow color zones as illustrated. As further shown, the tips are also color-coded. In addition thereto, the physical coding key 36 of each color tip 12 uniquely mates with the counterpart indicia 38 situated around each inlet port 26 (FIGS. 1 and 10B). In this manner, the kit 41 shown in FIG. 9B may be properly use by relatively untrained health-care workers operating in remote geographical regions where otherwise such medical diagnostics would not be available.

FIG. 10A is a across sectional detail elevation view of the dispenser tip 12 and optical analysis disc 18 of FIG. 9A illustrating association of the dispenser tip 12 with the disc just before loading of the liquid sample 32, stopper plug 14, and sealant 16. In FIG. 10B the assembly is shown with the liquid sample 32 loaded in the disc and the inlet port 26 sealed with the plug 14 seated on the annular lip portion 28 and sealant 16 deposited according.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

1. A liquid sample dispenser, comprising:
a body portion;
a tip portion connected to said body portion;
first and second retainer means moveably supported within said tip portion; and
a sealant contained between said first and second retainer means.

2. A liquid sample dispenser, comprising:
a body portion;
a tip portion connected to said body portion;
first and second retainer means moveably supported within said tip portion; and
a capsule pre-loaded with a sealant, placed between said first and second retainer means and releasable from said tip.

3. The dispenser according to claim 2 wherein said capsule is made of a material easily dissolving when released from the tip.

4. The dispenser according to claim 3 wherein said capsule is made of a material easily breaking when released from the tip.

5. A liquid sample dispenser, comprising:
a body portion;
a tip portion connected to said body portion;
first and second retainer means moveably supported within said tip portion; and
a sealant contained between said first and second retainer means, said tip portion including an operational end adapted to interact with an optical analysis disc having an inlet port and a channel layer for receiving a sample.

6. The dispenser according to claim 5 wherein said operational end includes coded indicia which corresponds to counterpart indicia associated with a respective inlet port in the optical analysis disc so that a sample in said tip portion may be loaded into a channel layer of the disc only through the respective inlet port having said counterpart indicia.

7. A liquid sample dispenser, comprising:
a body portion;
a tip portion connected to said body portion;
first and second retainer means moveably supported within said tip portion; and
a sealant contained between said first and second retainer means, said tip portion including a disinfectant.

8. The dispenser according to claim 7 wherein said disinfectant is an acid agent.

9. The dispenser according to claim 8 wherein said acid agent is aromatic in nature.

10. The dispenser according to claim 8 wherein said acid agent is selected for the group including triclosan, phenol, and trichlorophenol.

11. The dispenser according to claim 1, wherein said tip portion includes an operational end adapted to interact with an optical analysis disc.

12. The dispenser according to claim 7 wherein said operational end is open.

13. The dispenser according to claim 12 wherein said first retainer means is proximal to said operational end of said tip portion and includes a sealing plug adapted to seat in an inlet port of the optical analysis disc.

14. The dispenser according to claim 12 wherein said sealing plug is a rubber stopper.

15. The dispenser according to claim 11 wherein said second retainer means includes an upper stopper movable in said tip portion.

16. The dispenser according to claim 15 further comprising a stopping ring adapted to stop the movement of said upper stopper.

17. The dispenser according claim 1, wherein said sealant is an adhesive.

18. The dispenser according to claim 16 wherein said adhesive is a cyanoacrylate adhesive.

19. The dispenser according to claim 17 wherein said cyanoacrylate adhesive is formulated with lower boiling point solvents.

20. An optical analysis disc for use with the dispenser recited in claim 1.

21. An optical analysis disc for use with the dispenser recited in claim 2.

22. An optical analysis disc for use with the dispenser recited in claim 5.

23. An optical analysis disc for use with the dispenser recited in claim 7.

24. The optical analysis disc according to claim 20, wherein said tip portion of said dispenser includes a disinfectant.

25. An optical analysis disc, comprising:
a substrate;
a channel layer associated with said substrate;
a cover portion, said channel layer being positioned between said substrate and said cover portion; and
an inlet port formed in said cover portion adapted to receive a test sample and a sealing plug.

26. The optical analysis disc according to claim 25 wherein said inlet port is in fluid communication with said channel layer.

27. The optical analysis disc according to claim 26 wherein said inlet port has an annular lip portion adapted to receive a sealing plug.

28. The optical analysis disc according to claim 25 wherein said inlet port has a beak-away seal to protect pre-loaded chemistries in the channel layer.

29. An analysis kit, comprising:
a dispenser having a body portion, a tip portion connected to said body portion, first and second retainer means moveably supported within said tip portion, and a sealant contained between said first and second retainer means; and
an optical analysis disc including a substrate, a channel layer associated with said substrate, a cover portion located so that said channel layer is positioned between said substrate and said cover portion, and an inlet port formed in said cover portion adapted to receive a test sample and one of said retainer means.

30. The analysis kit according to claim 29 wherein said inlet port is in fluid communication with said channel layer.

31. The analysis kit according to claim 29 wherein said first retainer means is proximal to an open end of said tip portion and includes a sealing plug adapted to seat in said inlet port.

32. The analysis kit according to claim 29 wherein said tip portion of said dispenser includes an operational end adapted to interact with said optical analysis disc.

33. The analysis kit according to claim 32 wherein said operational end is open.

34. The analysis kit according to claim 32 wherein said first retainer means is proximal to said operational end of said tip portion and includes a sealing plug adapted to seat in said inlet port of said optical analysis disc.

35. The analysis kit according to claim 32 wherein said operational end includes coded indicia which corresponds to counterpart indicia associated with a respective inlet port in said optical analysis disc so that a sample in said tip portion may be loaded into said channel layer only through the respective inlet port having said counterpart indicia.

36. The analysis kit according to claim 29 wherein said tip portion of said dispenser is pre-loaded with a disinfectant.

37. A method for loading a biological sample into a analysis disc through a dispenser having a tip portion including retainer means and a sealant, said method comprising the following steps:
activating said dispenser for retrieving an pre-measured amount of said sample in said tip portion;
directing said tip portion to a respective disc having an inlet port in communication with a channel layer;
placing the tip portion within said inlet port;
activating said dispenser for moving the sample into the channel layer; and
sealing the inlet port.

38. The method according to claim 37 wherein said retainer means comprises a sealing plug adapted to be received in a annular lip portion of said inlet port.

39. The method according to claim 38 wherein said sealing step comprises a phase of seating said sealing plug within the inlet port.

40. The method according to claim 39 wherein said sealing step comprises a phase of releasing said sealant over said seated sealing plug.

41. The dispenser according to claim 1, where said sealent is provided in a capsule that is preloaded in said tip portion.

42. An analysis kit, comprising:
a set of tip portions adapted to be connected to a body portion of a dispenser, first and second retainer means moveably supported within each of said tip portions, and a sealant contained between said first and second retainer means; and
an optical analysis disc including a substrate, a channel layer including at least two analysis chambers, a cover portion located so that said channel layer and chambers are positioned between said substrate and said cover portion, and an inlet port formed in said cover portion adapted to receive a test sample and one of said retainer means.

43. The analysis kit according to claim 42 wherein said inlet port is in fluid communication with said channel layer.

44. The analysis kit according to claim 42 wherein said first retainer means is proximal to an open end of its respective tip portion and includes a sealing plug adapted to seat in said inlet port.

45. The analysis kit according to claim 42 wherein each of said tip portions includes an operational end adapted to interact with said optical analysis disc.

46. The analysis kit according to claim 45 wherein said operational end is open.

47. The analysis kit according to claim 45 wherein said first retainer means is proximal to said operational end of its respective tip portion and includes a sealing plug adapted to seat in said inlet port of said optical analysis disc.

48. The analysis kit according to claim 45 wherein said operational end of each tip portions includes coded indicia which corresponds to counterpart indicia associated with a respective inlet port in said optical analysis disc so that a sample in said tip portion may be loaded into said channel layer only through the respective inlet port having said counterpart indicia.

49. The analysis kit according to claim 42 wherein at least one of said tip portions is pre-loaded with a disinfectant.

50. The analysis kit according to claim 45 wherein said operational end of each tip portions includes coded indicia which corresponds to counterpart indicia associated with a respective inlet port in said optical analysis disc so that a sample in said tip portion may be loaded into a corresponding analysis chamber only through the respective inlet port having said counterpart indicia.

51. The analysis kit according to claim 50 wherein coded indicia and counterpart indicia are mating lock and key structures.

52. The analysis kit according to claim 42 wherein each of said at least two analysis chambers is associated with particularized indicia.

53. The analysis kit according to claim 52 wherein at least one of said set of tip portions includes particularized indicia matching said particularized indicia of one of said at least two analysis chambers, the matching tip portion and analysis chamber including compatible chemistries to perform a desired assay.

54. The analysis kit according to claim 52 wherein said particularized indicia is visual indicia.

55. The analysis kit according to claim 54 wherein said visual indicia include different color.

56. The analysis kit according to claim 54 wherein said visual indicia include different pictograms.

57. The analysis kit according to claim 54 wherein said visual indicia include written language in different fonts.

58. The analysis kit according to claim 54 wherein said visual indicia include written language in different colors.

59. The dispenser according to claim 12 wherein said second retainer means includes an upper stopper movable in said tip portion.

60. The dispenser according to claim 59 further comprising a stopping ring adapted to stop the movement of said upper stopper.

61. The dispenser according to claim 60 wherein said adhesive is a cyano-acrylate adhesive.
